(19) United States
(12) Patent Application Publication
(10) Pub. No.: US 2012/0101121 A1
(43) Pub. Date: Apr. 26, 2012

(54) DRUG DEMONSTRATING ANXIOLYTIC EFFECT BASED ON HYDROGENATED PYRIDO (4,3-B) INDOLES, ITS PHARMACOLOGICAL COMPOUND AND APPLICATION METHOD

(75) Inventors: Sergey Olegovich BACHURIN, Chernogolovka (RU); Taisiya Leonovna GARIBOVA, Nikolojamskiy (RU); Tatiana Alexandrovna VORONINA, Moscow (RU); Vladimir Viktorovich CRIGORIEV, Chernogolovka (RU)

(73) Assignee: Medivation Neurology ,Inc.

(21) Appl. No.: 12/667,035

(22) PCT Filed: Jun. 27, 2008
(86) PCT No.: PCT/US2008/008121

§ 371 Date: May 5, 2011

(30) Foreign Application Priority Data
Jun. 28, 2007 (RU) 2007124175

(51) Int. Cl. A61K 31/437; A61P 25/22; A61P 25/00; C07D 471/04

(52) U.S. Cl. 514/292; 546/85; 546/87

(57) ABSTRACT

Compositions based on hydrogenated pyrido[4,3-b]indoles (variants) of formula (1) or formula (2): are provided, as are methods and kits using those compositions for the treatment of anxiety or mood disorders characterized by stresses, anxiety, neuroses, obsessive fears and their consequences.

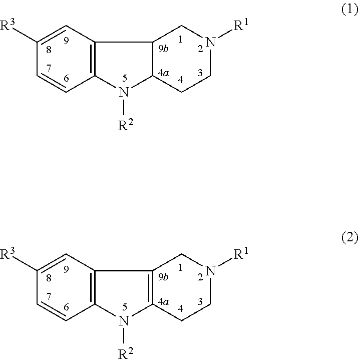

### CROSS-REFERENCE TO RELATED APPLICATIONS

This application claims priority to Russian Patent Application No. 2007124175, filed Jun. 28, 2007, which is incorporated herein by reference in its entirety.

### STATEMENT OF RIGHTS TO INVENTIONS MADE UNDER FEDERALLY SPONSORED RESEARCH

Not applicable.

### TECHNICAL FIELD

The invention relates to the field of medicine, and more specifically, to application of chemical compounds for the purpose of creating novel anxiolytic drugs for treatment and prevention of stresses, anxiety, neuroses, obsessive fears and their consequences.

### BACKGROUND OF THE INVENTION

At the present time, because of an increase in the number of psycho-traumatic and stress-causing factors, the accelerating pace of modern life, an intensification of labor, an increase in information flow, ecological problems, natural disasters, and the like, there is a sharp increase in the number of patients suffering from neurotic and neurosis-like conditions, accompanied by anxiety, fears, increased emotional lability, which are defined in psychiatry as boundary conditions proceeding without pronounced mental defects and cerebral degeneration. Emotional disorders are also observed during chronic somatic diseases. In economically developed countries, at least 80% of the group of mental diseases is represented by neurotic diseases, and 10-12% of the healthy population suffers from neuroses (Arushanyan, E. B., ANXIOLYTIC AGENTS (in Russian), Stavropol, Stavropol Medical Academy, 2001, p. 240).

Currently, stress, anxiety and fears are commonly treated with compounds called “anxiolytics.” Anxiolytics are drugs capable of reducing pathological data. Compounds of the benzodiazepine series are used the most, among which Diazepam® (Seduxen®, Valium®) is used as a reference compound. However, benzodiazepine compounds including Diazepam® have significant side effects. Therapeutic doses of those drugs cause sedation, muscle relaxation, memory impairment and pose a risk of developing drug dependence. (Register of Drugs in Russia (in Russian), ENCYCLOPEDIA OF DRUGS, V. 14., edited by G. L. Vyshkovskiy, Moscow, RLS-2006). Consequently, there is an ongoing search for a new generation of anxiolytic agents free of side effects typical for benzodiazepine tranquilizers.

There remains a significant medical need for additional or alternative therapies for treatment of anxiety or mood disorders characterized by stresses, anxiety, neuroses, or obsessive fears. Preferably, the therapeutic agents can improve the quality of life, relieve the stresses, anxieties, neuroses, and obsessive fears of patients suffering from such disorders.

Therefore, the task this invention is to solve is to expand the range of available agents for use as novel anxiolytic agents, i.e., effective compounds for the treatment and prevention of stresses, anxiety, neuroses, obsessive fears, and their consequences.

### DETAILED DESCRIPTION OF THE INVENTION

### Definitions

As used herein, unless clearly indicated otherwise, the terms “a,” “an,” and the like refer to one or more. It is also understood and clearly conveyed by this disclosure that reference to “the compound” or “a compound” includes and refers to any compound or pharmaceutically acceptable salt or other form thereof as described herein, for example, a hydrogenated pyrido[4,3-b]indole, such as the compound dimebon.

As used herein, the terms “anxiety disorder,” “mood disorder,” or “anxiety or mood disorder” refer to several different forms of abnormal, pathological anxiety, fears, and phobias encompassing psychiatric disorders of the nervous system based on stress, anxiety, or worry not based on fact. Anxiety disorders include generalized anxiety disorder, panic disorder, phobias, social anxiety disorder, obsessive-compulsive disorder, post-traumatic stress disorder, and separation anxiety. Such disorders encompass anxiety, fears, and phobias related to or accompanying psychiatric conditions or disorders, specifically excluding anxiety caused by or related to trauma arising from ischemia, hemorrhagic insult (i.e., ischemic or hemorrhagic stroke), traumatic brain injury or resulting from underlying disease conditions accompanied by mental defects and/or cerebral or other neurodegeneration such as Alzheimer's disease, Huntington's disease, amyotrophic lateral sclerosis, Parkinson's disease, multiple sclerosis, schizophrenia, age-associated memory impairment, mild cognitive impairment, canine cognitive dysfunction syndrome, autism, autism spectrum disorder, Asperger syndrome, and Rett syndrome.

As used herein, the term “generalized anxiety disorder” refers to a common chronic anxiety or mood disorder that affects twice as many women as men and can lead to considerable impairment. As the name implies, generalized anxiety disorder is characterized by long-lasting anxiety that is not focused on any particular object or situation, i.e., it is unspecific or free-floating.

As used herein, the term “panic disorder” refers to a anxiety or mood disorder characterized by brief attacks of intense terror and apprehension or sudden bouts of intense anxiety that cause trembling and shaking, confusion, dizziness, nausea, difficulty breathing, and feelings of impending doom or a situation that would be embarrassing. Panic disorder can be diagnosed when several apparently spontaneous attacks lead to a persistent concern about future attacks.

As used herein, the term “phobia” refers to a class of anxiety or mood disorders characterized by a strong, irrational fear and avoidance of particular objects or situations. The person knows the fear is irrational, yet the anxiety remains. Phobic disorders differ from generalized anxiety disorders and panic disorders because there is a specific stimulus or situation that elicits a strong fear response.

As used herein, the term “social anxiety disorder” or “social phobia” refers to an anxiety or mood disorder characterized by intense fear of being negatively evaluated by others or of being publicly embarrassed because of impulsive acts. Almost everyone experiences “stage fright” when speaking or performing in front of a group, but people suffering from social anxiety disorder tend to become so anxious that speaking or performing in public is out of the question, sometimes to the point that normal life can become impossible.

As used herein, the term “obsessive-compulsive disorder” or “OCD” refers to a type of anxiety or mood disorder primarily characterized by obsessions and/or compulsions. The term “obsession” refers to generally distressing, repetitive, intrusive thoughts or images that the individual often realizes are senseless. The term “compulsion” refers to repetitive behaviors that the person feels forced or compelled to do, often to relieve anxiety.

As used herein, the term “post-traumatic stress disorder” refers to an anxiety or mood disorder which results from a traumatic experience. Post-traumatic stress can result from an extreme situation, such as being involved in warfare, rape, a hostage situation, or a serious accident. It can also result from chronic exposure to a severe stressor, for example, soldiers who endure individual battles but cannot cope with an unending sequence of battles. The sufferer may experience flashbacks, avoidance behavior, and other symptoms.

As used herein, the term “separation anxiety” refers to an anxiety or mood disorder characterized by the feeling of excessive and inappropriate levels of anxiety over being separated from an attachment figure or from a person or place that gives a feeling of safety. While it most commonly observed in children, for example, on being left at school by a parent, it is sometimes also observed in adolescents and adults. Separation anxiety itself is a normal part of development in babies or children, but can be considered an anxiety disorder when the feeling of anxiety is excessive.

As used herein, the term “anxiolytic” refers to drug compounds used to treat symptoms of patients having anxiety or mood disorders, including stress, anxiety, neuroses, and obsessive fears. Anxiolytics are generally divided into two broad categories: benzodiazepines and non-benzodiazepines. Benzodiazepines are typically prescribed for short-term relief of severe and disabling anxiety, or for latent periods associated with other medications commonly prescribed to treat an underlying anxiety disorder. Commonly prescribed benzodiazepines include lorazepam (Ativan®), clonazepam (Klonopin®), alprazolam (Xanax®), and diazepam (Valium®). Potential drawbacks to use of benzodiazepines include the accompanying sedation, muscle relaxation, and memory impairment, as well as the risk of developing drug dependence. Non-benzodiazepines include serotonin 1A agonists, such as Buspirone®, which lacks the sedation and potential dependence associated with benzodiazepines, and causes much less cognitive impairment; barbiturates and meprobamate, which exert an anxiolytic effect linked to the sedation they cause, though the risk of abuse and addiction is high; and a host of herbal remedies purportedly have anxiolytic effect, including valerian root, kava, chamomile, and Blue Lotus extracts, though little evidence of efficacy exists.

As used herein, unless clearly indicated otherwise, the term “an individual” refers to a mammal, including but not limited to a human, bovine, primate, equine, canine, feline, porcine, and ovine animals. Thus, the invention finds use in both human medicine and in the veterinary context, including use in agricultural animals and domestic pets. The individual may be a human who has been diagnosed with or is suspected of having an anxiety or mood disorder characterized by stresses, anxiety, neuroses, or obsessive fears. The individual may be a human who exhibits one or more symptoms associated with an anxiety or mood disorder characterized y stresses, anxiety, neuroses, or obsessive fears. The individual may be a human who has a mutated or abnormal gene associated with elevated risk of an anxiety or mood disorder, but who has not been diagnosed with such a disease. The individual may be a human who is genetically or otherwise predisposed to developing an anxiety or mood disorder.

As used herein, an “at risk” individual is an individual who is at risk of developing or suffering an anxiety or mood disorder characterized by stresses, anxiety, neuroses, or obsessive fears. An individual “at risk” may or may not have detectable disease, and may or may not have displayed detectable disease prior to the treatment methods described herein. “At risk” denotes that an individual has one or more so-called risk factors, which are measurable parameters that correlate with likelihood of experiencing an anxiety or mood disorder characterized by stresses, anxiety, neuroses, or obsessive fears. An individual having one or more of these risk factors has a higher probability of suffering such a disorder than an individual without those risk factor(s). Risk factors include, but are not limited to, age, sex, race, diet, history of previous disease or injury, presence of precursor disease or injury, genetic (i.e., hereditary) considerations, and environmental exposure. Individuals at risk for an anxiety or mood disorder characterized by stresses, anxiety, neuroses, or obsessive fears include, e.g., those having relatives who have experienced such diseases, and those whose risk is determined by analysis of genetic or biochemical markers.

As used herein, the term “pharmaceutically active compound,” “pharmacologically active compound” or “active ingredient” refers to a chemical compound, for example, a hydrogenated pyrido[4,3-b]indole such as dimebon, that induces a desired effect, e.g., treating and/or preventing and/or delaying the onset or severity of anxiety or mood disorders characterized by stresses, anxiety, neuroses, or obsessive fears.

As used herein, the term “pharmacological means” or “pharmaceutical formulation” refers to the use of any therapeutic dosage form, including immediate or sustained release forms, containing a compound, e.g., a hydrogenated pyrido[4,3-b]indole such as dimebon, or a compound of formula (1) or formula (2), which may find prophylactic or therapeutic use in medicine for the treatment of anxiety or mood disorders characterized by stresses, anxiety, neuroses, or obsessive fears. Such means or formulations may also contain pharmaceutically acceptable excipients, including preservatives, solubilizers, stabilizers, re-wetting agents, emulgators, sweeteners, dyes, adjusters, salts for the adjustment of osmotic pressure, buffers, coating agents or antioxidants.

As used herein, the term “pharmaceutically acceptable” or “pharmacologically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, the term “effective amount” refers to the use of that amount of compound, e.g., a compound of formula (1) or formula (2) which in combination with its activity and toxicity characteristics, and also on the basis of the knowledge of a specialist, should be effective in a given therapeutic form.

As used herein, the term “therapeutically effective amount” refers to an amount of a compound or a combination therapy sufficient to produce a desired therapeutic outcome (e.g., reducing the severity or duration of, stabilizing the severity of, or eliminating one or more symptoms associated with anxiety or mood disorders characterized by stresses, anxiety, neuroses, or obsessive fears). For therapeutic use, beneficial or desired results include, e.g., clinical results such as reducing or eliminating stress, anxiety, neuroses, or obsessive fears, improving mood or otherwise reversing symptoms of the disorder, decreasing one or more biochemical, histologic and/or behavioral symptoms associated with the disorder, including associated complications and intermediate pathological phenotypes presenting during development or progression of the anxiety or mood disorder, increasing the quality of life of those suffering such diseases, decreasing the dose of other medications required to treat the anxiety or mood disorder, enhancing the effect of another medication, and/or prolonging survival of patients.

A “prophylactically effective amount” refers to an amount of a compound or a combination therapy sufficient to prevent or reduce the severity of one or more future symptoms of anxiety or mood disorders when administered to an individual who is _{—} susceptible and/or who may develop such a disorder. For prophylactic use, beneficial or desired results include, e.g., clinical results such as reducing or eliminating stress, anxiety, neuroses, or obsessive fears, improving mood or otherwise reversing symptoms of the disorder, decreasing one or more biochemical, histologic and/or behavioral symptoms associated with the disorder, including associated complications and intermediate pathological phenotypes presenting during development or progression of the anxiety or mood disorder, increasing the quality of life of those suffering such diseases, decreasing the dose of other medications required to treat the anxiety or mood disorder, enhancing the effect of another medication, and/or prolonging survival of patients.

As used herein, “treatment” or “treating” is an approach for obtaining beneficial or desired results, including clinical results. For purposes of this invention, beneficial or desired clinical results include, but are not limited to, one or more of the following: decreasing one more symptoms resulting from anxiety or mood disorders, limiting the extent of disability resulting from anxiety or mood disorders, increasing the quality of life, and/or decreasing the dose of one or more other medications required to treat such diseases. In some embodiments, an individual or combination therapy of the invention reduces the severity of one or more symptoms associated with anxiety or mood disorders by at least 10, 20, 30, 40, 50, 60, 70, 80, 90, or 95% compared to the corresponding symptom in the same subject prior to treatment or compared to the corresponding symptom in other subjects not receiving the therapy.

As used herein, the term “combination therapy” includes a first therapy, for example, one or more hydrogenated pyrido[4,3-b]indoles (e.g., dimebon) or pharmaceutically acceptable salts thereof, in conjunction with a second therapy that includes one or more other compounds (or pharmaceutically acceptable salts thereof) or therapies (e.g., surgical procedures) useful for decreasing one more symptoms resulting from anxiety or mood disorders, limiting the extent of disability resulting from such disorders, increasing the quality of life, decreasing the dose of one or more other medications required to treat the disease, and/or prolonging survival time for individuals suffering from such diseases. Administration in “conjunction with” another compound includes administration in the same or different composition, either sequentially, simultaneously, or continuously using the same or different route of administration for each compound. In some variations, the combination therapy optionally includes one or more pharmaceutically acceptable carriers or excipients, non-pharmaceutically active compounds, and/or inert substances.

As used herein, the term “simultaneous administration” includes a first therapy and a second or subsequent therapy in a combination therapy that are administered, for example, with a time separation of no more than about 15 minutes, such as no more than about any of 10, 5, or 1 minutes. When the compounds are administered simultaneously, the first and second therapies may be contained in the same composition or in separate compositions.

As used herein, the term “sequential administration” includes first therapy and second or subsequent therapy in a combination therapy administered, for example, with a time separation of more than about 15 minutes, such as more than about any of 20, 30, 40, 50, 60 minutes, or more than about any of 1 hour to about 24 hours, about 1 hour to about 48 hours, about 1 day to about 7 days, about 1 week to about 4 weeks, about 1 week to about 8 weeks, about 1 week to about 12 weeks, about 1 month to about 3 months, or about 1 month to about 6 months. Either the first therapy or the second or subsequent therapy may be administered first. The first and second therapies are contained in separate compositions, which may be contained in the same or different packages or kits. The invention embraces the sequential administration of all combinations described herein.

Thus, an effective amount of a combination therapy includes an amount of the first therapy and an amount of the second therapy that when administered sequentially, simultaneously, or continuously produces a desired outcome. Suitable doses of any of the co-administered compounds may optionally be lowered due to the combined action (e.g., additive or synergistic effects) of the compounds. In various embodiments, treatment with the combination of the first and second therapies may result in an additive or even synergistic (e.g., greater than additive) result compared to administration of either therapy alone. In some embodiments, a lower amount of each pharmaceutically active compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a pharmaceutically active compound in a combination therapy than the amount generally used for individual therapy. Preferably, the use of a small amount of pharmaceutically active compound results in a reduction in the number, severity, frequency, or duration of one or more side-effects associated with the compound.

As is understood in the clinical context, an effective dosage of a drug, compound or pharmaceutical composition containing a compound described by the invention, e.g., a hydrogenated pyrido[4,3-b]indole such as dimebon, or a compound of the formula (1) or (2) or any compound described herein (e.g., any of compounds 1 to 9) may be achieved in conjunction with another drug, compound or pharmaceutical composition.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (i.e., pH-dependent or non-pH-dependent solubility, different degrees of water solubility, and the like) and formulating the mixture according to the desired route of delivery (i.e., coated capsules, implantable reservoirs, injectable solutions containing biodegradable capsules, and the like).

For use herein, unless clearly indicated otherwise, the term “sustained release system” (also referred to as “a system” or “the system”) refers to a drug delivery system capable of sustaining the rate of delivery of a compound to an individual for a desired duration, which may be an extended duration. A desired duration may be any duration that is longer than the time required for a corresponding immediate-release dosage form to release the same amount (e.g., by weight or by moles) of compound, and can be hours or days. A desired duration may be at least the drug elimination half life of the administered compound and may be about any of, e.g., at least about 6 hours, or at least about 12 hours, or at least about 24 hours, or at least about 30 hours, or at least about 48 hours, or at least about 72 hours, or at least about 96 hours, or at least about 120 hours, or at least about 144 or more hours, and can be at least about one week, at least about 2 weeks, at least about 3 weeks, at least about 4 weeks, at least about 8 weeks, at least about 16 weeks or more.

### Exemplary Indications

Provided herein are methods and compositions for the treatment of anxiety or mood disorders characterized by abnormal, pathological anxiety, fears, and phobias encompassing psychiatric disorders of the nervous system based on stress, anxiety, or worry not based on fact. Anxiety disorders include generalized anxiety disorder, panic disorder, phobias, social anxiety disorder, obsessive-compulsive disorder, post-traumatic stress disorder, and separation anxiety.

Anxiety can be an unpleasant emotional state frequently accompanied by physiological symptoms that may lead to fatigue and/or exhaustion. Fear can be an emotional and physiological response to a recognized threat, whether external or internal. Because fear of recognized threats causes unpleasant mental and physical changes similar to those associated with anxiety, the terms fear and anxiety are sometimes used interchangeably. Phobias are characterized by persistent or irrational fear or anxiety, such as anxiety about being in a place or situation where escape is difficult or embarrassing (i.e., agoraphobia). Anxiety disorders can manifest as debilitating chronic conditions present from an early age or can begin suddenly after a triggering event, and are frequently prone to flare up at times of high stress. Such disorders often manifest with physical symptoms as well. For example, anxiety can be accompanied by headaches, sweating, palpitations, and hypertension.

Individuals having generalized anxiety disorder feel afraid of something but generally cannot articulate the specific fear. Consequently, they fret constantly and have a hard time controlling their worries. Because of persistent muscle tension and autonomic fear reactions, they may develop headaches, heart palpitations, dizziness, insomnia, and chest pains. These physical symptoms, combined with the intense, long-term anxiety, can make it very difficult for affected individuals to cope with normal daily activities.

Panic attacks encompass abruptly arising fear or discomfort that peaks in a very short time (sometimes 10 minutes or less), but that occasionally persists for hours. Although panic attacks sometimes seem to occur out of nowhere, they generally happen after frightening experiences, prolonged stress, or even exercise.

Unlike generalized anxiety and panic disorders, phobic disorders are often triggered by a specific stimulus or situation that elicits a strong fear response. People with phobias tend to have especially powerful imaginations, so they vividly anticipate terrifying consequences from encountering such feared objects as knives, bridges, blood, enclosed places, certain animals or situations. Such individuals generally recognize that those fears are excessive and unreasonable but usually cannot control their anxiety.

Obsessive-compulsive disorder (“OCD”) refers to a type of anxiety or mood disorder primarily characterized by obsessions and/or compulsions. In many cases, the connection between an obsession and the associated compulsive behavior may appear illogical (e.g., a compulsion of walking in a certain pattern might be used to alleviate an obsession that something bad is about to happen). Occasionally the motivation for a particular compulsion cannot be readily explained: it may simply be an urge to complete a particular ritual triggered by nervousness.

Exemplary hydrogenated pyrido[4,3-b]indoles

This task is solved by using hydrogenated pyrido[4,3-b]indoles described by formula (1) or formula (2) as anxiolytic agents.

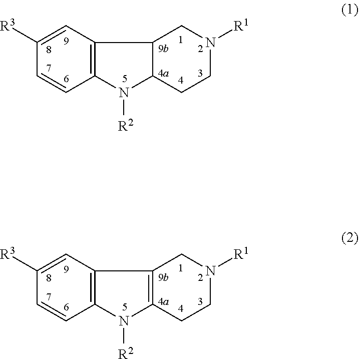

In case of using compounds described by formula (1), R¹ is selected from the group consisting of CH₃—, CH₃CH₂— or PhCH₂—; R² is selected from the group consisting of H—, PhCH₂— or 6-CH₃-3-Py-(CH₂)₂-; R³ is selected from the group consisting of H—, CH₃— or Br—. Described compounds also include salts with pharmaceutically acceptable acids.

One of the compounds which can be used as an anxiolytic agent, can be a compound described by formula (1), where R¹ corresponds to CH₃—, R² corresponds to H—, and R³ corresponds to CH₃—. The structures as drawn in formula (1) and formula (2) embrace all stereoisomers.

In case of compounds described by formula (2), R¹ is selected from the group consisting of CH₃—, CH₃CH₂— or PhCH₂—; R² is selected from the group consisting of H—, PhCH₂— or 6-CH₃-3-Py-(CH₂)₂-; and R³ is selected from the group consisting of H—, CH₃— or Br—. Described compounds may represent salts with pharmaceutically acceptable acids.

One of the compounds that can be used as an anxiolytic agent and employed for treatment and prevention of stresses, anxiety, neuroses, obsessive fears and their consequences, can be the compound described by formula (2), where R¹ corresponds to CH₃CH₂— or PhCH₂—; R² corresponds to H-; and R³ corresponds to H—; or the compound where R¹ corresponds to CH₃—; R² corresponds to PhCH₂—, and R³ corresponds to CH₃—; or the compound, where R¹ corresponds to CH₃—, R² corresponds to 6-CH₃-3-Py-(CH₂)₂-, and R³ corresponds to H—; or the compound, where R¹ corresponds to CH₃—, R² corresponds to 6-CH₃-3-Py-(CH₂)₂-, and R³ corresponds to CH₃—; or the compound, where R¹ corresponds to CH₃—, R² corresponds to H—, and R³ corresponds to H—; or the compound, where R¹ corresponds to CH₃—, R² corresponds to H—, and R³ corresponds to Br—.

Any of the above-described compounds can be used as an anxiolytic agent for treatment and prevention of anxiety or mood disorders accompanied by stresses, anxiety, neuroses, obsessive fears and their consequences.

Hydrogenated pyrido[4,3-b]indoles described by formula (1) or formula (2) are well-known compounds which are widely used in pharmacological practice. Extensive studies have been conducted pertaining to a number of known compounds, which represent derivatives of tetra- and hexahydro-1H-pyrido[4,3-b]indole (hereinafter all compounds are described by formula (1) and (2) and demonstrate a wide spectrum of biological activity). In the series of 2,3,4,5-tetrahydro-1H-pyrido[4,3-b]indoles, the following types of activity were found: antihistamine (OS-DE No. 1813229, Dec. 6, 1968; No. 1952800, Oct. 20, 1969), central-depressant, anti-inflammation (U.S. Pat. No. 3,718,657, Dec. 13, 1970), neuroleptic (Herbert C. A., Plattner S. S., Wehch W. N., Mol. Pharm., 1980, v. 17, N I, p. 38-42) and others. Derivatives of 2,3,4,4a,5,9b-hexahydro-1H-pyrido[4,3-]indole demonstrate psychotropic (Welch W. H., Herbert C. A., Weissman A., Koe K. B., J. Med. Chem., 1986, vol. 29, N 10, p. 2093-2099), anti-aggressive, anti-arrhythmic and other types of activities.

All the compounds described above are known from the literature and include the following specific compounds:
1. Cis(±)2,8-dimethyl-2,3,4,4a,5,9b-hexahydro-1H-pyrido[4,3-b]indole and its dihydrochloride;
2. 2-ethyl-2,3,4,5-tetrahydro-1H-pyrido[4,3-b]indole;
3. 2-benzyl-2,3,4,5-tetrahydro-1H-pyrido[4,3-b]indole;
4. 2,8-dimethyl-5-benzyl-2,3,4,5-tetrahydro-1H-pyrido[4,3-b]indole and its hydrochloride;
5. 2-methyl-5-[2-(6-methyl-3-pyridyl)ethyl]-2,3,4,5-tetrahydro-1H-pyrido[4,3-b]indole and its sesquisulphate monohydrate;
6. 2,8-dimethyl-5-[2-(6-methyl-3-pyridyl)ethyl]-2,3,4,5-tetrahydro-1H-pyrido[4,3-b]indole and its dihydrochloride (dimebon);
7. 2-methyl 2,3,4,5-tetrahydro-1H-pyrido[4,3-b]indole;
8. 2,8-dimethyl-2,3,4,5-tetrahydro-1H-pyrido[4,3-b]indole and its methyliodide; and
9. 2-methyl-8-bromine-2,3,4,5-tetrahydro-1H-pyrido[4,3-b]indole and its hydrochloride.

Preparation and neuroleptic properties of the compound 1 are known, for example, from the publication: Yakhontov, L. N. and Glushkov, R. G. Synthetic drugs (in Russian; edited by A. G. Natradze, Moscow, Meditsyna, 1983, pp. 234-237). Preparation of the compounds 2, 8 and 9, as well as information about their properties as serotonin antagonists, are described, for example, in C. J. Cattanach, A. Cohen and B. H. Brown in J. Chem. Soc. (series C), 1968, 1235-1243. Synthesis of the compound 3 is described, for example, in the article: N. P. Buu-Hoi, O. Roussel, and P. Jacquignon, J. Chem. Soc., 1964, no. 2, pp. 708-711. In “General Chemistry” (in Russian), 1956, vol. 26, pp. 3149-3154, N. F. Kucherova and N. K. Kochetkov described synthesis of the compound 4, and preparation of the compounds 5 and 6 are known, for example, from an article by A. N. Kost, M. A. Yurovskaya, and T. V. Mel'nikova in “Chemistry of heterocyclic compounds” (in Russian), 1973, No. 2, pp. 207-212. Publications by U. Horlein, Chem. Ber., 1954, Bd. 87, hft. 4, pp. 463-472 describe synthesis of compound 7. M. A. Yurovskaya and I. L. Rodionov in “Chemistry of heterocyclic compounds”, 1981, No. 8, pp. 1072-1078 describe preparation of the methyliodide of the compound 8.

Several drugs are produced based on derivatives of tetra- and hexahydro-1H-pyrido[4,3-b]indole: Diazolin® (mebhydroline), Carbidine® (dicarbine), Stobadin®, Gevotroline®, Diazolin® (2-methyl-5-benzyl-2,3,4,5-tetra-hydro-IH-pyrido[4,3-b]indole)dihydrochloride (Klyuev, M. A., “Drugs used in the medical practice of the USSR, (in Russian)—Moscow, Meditsyna 1991, p. 512) and Dimebon (2,8-dimethyl-5-[2-(6-methyl-pyridyl-3)ethyl]-2,3,4,5-tetrahydro-1H-pyrido[4,3-b]indole dihydrochloride (Mashkovskiy, M. D., “Medicinal drugs (in 2 parts),” (in Russian), part 1, 12^{th} edition, Moscow, Meditsyna, 1993, p. 383), as well as its close analog Dorastine (2-methyl-8-chlorine-5-[2-(6-methyl-3-pyridypethyl]-2,3,4,5-tetrahydro-1H-pyrido[4,3-b]indole) dihydrochloride (USAN and USP dictionary of drugs names (United States Adopted Names 1961-1988, U.S. Pharmacopeia and National Formulary for Drugs, and other nonproprietary drug names), 1989, 26th Edition, pg. 196) are known as antihistamine compounds. Carbidine® (dicarbidine) (dihydrochloride cis(±)-2,8-dimethyl-2,3,4,4a,5,9b-hexahydro-1H-pyrido[4,3-b]indole) is a domestic neuroleptic with antidepressant effect (Yakhontov, L. N., Glushkov, R. G., “Synthetic medicinal drugs,” (in Russian)(edited by A. G. Natradze, Moscow, Meditsyna, 1983, pp. 234 -237), and its (−)-isomer, Stobadin®, are known as anti-arrhythmic drugs (Kitlova, M., Gibela, P., Drimal, J., BRATISI. LEK. LISTY, 1985, 84(5):542-546).

In recent years, it was found that derivatives of hydrogenated pyrido[4,3-b]indoles described by formula (1) or (2) and, specifically, Dimebon, can affect two major subtypes of ionotropic glutamate receptors of the CNS in mammals—AMPA and NMDA receptors. That property enables their use as drugs for treatment of Alzheimer's disease as well as geroprotector agents. Dimebon potentiates transmembrane currents caused by activation of the AMPA receptors, while at the same time blocking NMDA receptors (V. V. Grigoryev, O. A. Dranyi, and C. O. Bachurin, “Comparative study of the mechanisms of Dimebon and Memantine effect on AMPA and NMDA-subtypes of glutamate receptors of the cerebral neurons in rats,” (in Russian), Bull. Experim. Biol. Med., 2003, No. 11, pp. 535-538). See also the following patents and patent publications: U.S. Pat. Nos. 6,187,785 and 7,021,206, International Publication Nos. WO 2005/055951, WO 2007/020516, and WO 2007/087425, PCT Application No. PCT/US2007/022645, U.S. Patent Publication Nos. 2007-0117834-A1 and 2007-0117835-A1.

To their surprise, the inventors have found that the compounds described by formula (1) and formula (2) demonstrate anxiolytic effect due to new properties discovered in them that were not expected from the chemical structure of these compounds or from earlier known properties (specifically, positive modulators of AMPA receptors or blockers of NMDA receptors), and can be used as anxiolytic drugs.

According to the invention, pharmacological drugs demonstrating anxiolytic effect and containing an active compound and a pharmaceutically acceptable vehicle as an active compound comprise an effective amount of a hydrogenated pyrido[4,3-b]indole such as dimebon, including compounds described by the formula (1) or formula (2). The term “pharmacological drug” refers to utilization of any drug formulation containing compounds of formula (1) or formula (2), which can be used as anxiolytic drugs for prevention or treatment of anxiety or mood disorders characterized by stresses, anxiety, neuroses, obsessive fears and their consequences.

Provided herein are methods of using hydrogenated pyrido[4,3-b]indoles of formula (1) or pharmaceutically acceptable salts thereof to treat anxiety or mood disorders:

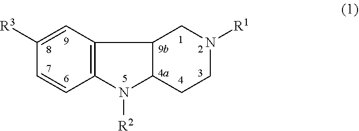

In certain embodiments, R¹ is selected from the group consisting of CH₃—, CH₃CH₂— and PhCH₂—; R² is selected from the group consisting of H—, PhCH₂—, and 6-CH₃-3-Py-(CH₂)₂-; and R³ is selected from the group consisting of H, CH₃— and Br—. In certain embodiments, R¹ corresponds to CH₃—, R² corresponds to H—, and R³ corresponds to CH₃—. In certain embodiments, the compound is a salt of a pharmaceutically acceptable acid. In certain embodiments, the anxiety or mood disorder is selected from the group consisting of generalized anxiety disorder, panic disorder, phobias, social anxiety disorder, obsessive-compulsive disorder, post-traumatic stress disorder, and separation anxiety.

Also provided herein are methods of using hydrogenated pyrido[4,3-b]indoles of formula (2) or pharmaceutically acceptable salts thereof as anxiolytic agents:

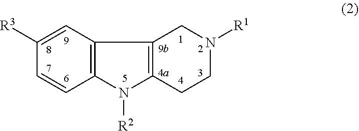

In certain embodiments, R¹ is selected from the group consisting of CH₃—, CH₃CH₂— and PhCH₂—; R² is selected from the group consisting of H—, PhCH₂—, and 6-CH₃-3-Py-(CH₂)₂-; and R³ is selected from the group consisting of H, CH₃— and Br—. In certain embodiments, R¹ corresponds to CH₃CH₂— or PhCH₂—; R² corresponds to H—; and R³— corresponds to H—. In certain embodiments, R¹ corresponds to CH₃—; R² corresponds to PhCH₂—; and R³ corresponds to CH₃—. In certain embodiments, R¹ corresponds to CH₃—, R² corresponds to 6-CH₃-3-Py-(CH₂)₂-, and R³— corresponds to H—. In certain embodiments, R¹ corresponds to CH₃—, R² corresponds to 6-CH₃-3-Py-(CH₂)₂-, and R³— corresponds to CH₃—. In certain embodiments, R¹ corresponds to CH₃—, R² corresponds to PhCH₂—, and R³ corresponds to CH₃—. In certain embodiments, R¹ corresponds to CH₃—, R² corresponds to H—, and R³ corresponds to Br—. In certain embodiments, the compound is a salt of a pharmaceutically acceptable acid. In certain embodiments, the compound is 2,8-dimethyl-5-[2-(6-methyl-pyridyl-3)-ethyl]-2,3,4,5-tetrahydro-1H-pyrido[4,3-b]indole (Dimebon). In certain embodiments, the anxiety or mood disorder is selected from the group consisting of generalized anxiety disorder, panic disorder, phobias, social anxiety disorder, obsessive-compulsive disorder, post-traumatic stress disorder, and separation anxiety.

Further provided herein are pharmaceutical compositions having anxiolytic effect, comprising an active compound and a pharmaceutically acceptable carrier, wherein the active compound comprises an effective amount of a compound of formula (1) or formula (2):

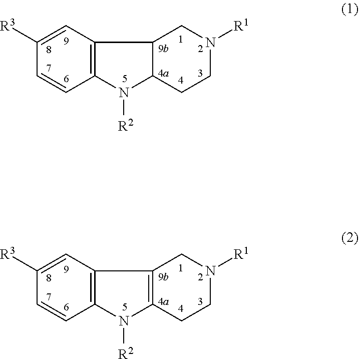

In certain embodiments, R¹ is selected from the group consisting of CH₃—, CH₃CH₂— and PhCH₂—; R² is selected from the group consisting of H—, PhCH₂—, and 6-CH₃-3-Py-(CH₂)₂-; and R³ is selected from the group consisting of H, CH₃— and Br—. In certain embodiments, the compound is 2,8-dimethyl-5-[2-(6-methyl-pyridyl-3)-ethyl]-2,3,4,5-tetrahydro-1H-pyrido[4,3-b]indole (Dimebon).

Also provided herein are methods of treating and preventing anxiety or mood disorders, comprising administering to the patient a composition containing an effective amount of a compound of formula (1) or formula (2):

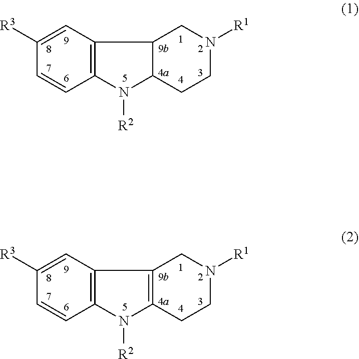

In certain embodiments, R¹ is selected from the group consisting of CH₃—, CH₃CH₂— and PhCH₂—; R² is selected from the group consisting of H—, PhCH₂—, and 6-CH₃-3-Py-(CH₂)₂-; and R³ is selected from the group consisting of H, CH₃— and Br—. In certain embodiments, the effective amount is administered at a dose between 0.1 mg/kg and 10 mg/kg of body weight at least once a day for a duration necessary to achieve therapeutic effect. In certain embodiments, the anxiety or mood disorder is selected from the group consisting of generalized anxiety disorder, panic disorder, phobias, social anxiety disorder, obsessive-compulsive disorder, post-traumatic stress disorder, and separation anxiety. In certain embodiments, the compound is 2,8-dimethyl-5-[2-(6-methyl-pyridyl-3)-ethyl]-2,3,4,5-tetrahydro-1H-pyrido[4,3-b]indole (Dimebon).

In any of the above embodiments, the hydrogenated pyrido[4,3-b]indole of formula (2) is 2,8-dimethyl-5-[2-(6-methyl-pyridyl-3)-ethyl]-2,3,4,5-tetrahydro-1H-pyrido[4,3-b]indole (Dimebon), and the anxiety or mood disorders are related to or accompanying psychiatric conditions or disorders. In any of the above embodiments, the hydrogenated pyrido[4,3-b]indole of formula (2) is 2,8-dimethyl-5-[2-(6-methyl-pyridyl-3)-ethyl]-2,3,4,5-tetrahydro-1H-pyrido[4,3-b]indole (Dimebon), and the anxiety or mood disorders are not caused by or related to trauma arising from ischemia, hemorrhagic insult (i.e., ischemic or hemorrhagic stroke), traumatic brain injury or resulting from underlying disease conditions accompanied by mental defects and/or cerebral or other neurodegeneration such as Alzheimer's disease, Huntington's disease, amyotrophic lateral sclerosis, Parkinson's disease, multiple sclerosis, schizophrenia, age-associated memory impairment, mild cognitive impairment, canine cognitive dysfunction syndrome, autism, autism spectrum disorder, Asperger syndrome, and Rett syndrome.

### Exemplary Formulations

One or more compounds of formula (1) or formula (2) can be used in the preparation of a formulation, such as a pharmaceutical formulation, by combining the compound or compounds as active ingredient with a pharmaceutically acceptable carrier, which are known in the art. See, e.g., Remington's Pharmaceutical Sciences, 20th ed. (2000), Mack Publishing Co., Philadelphia, Pa., which is incorporated herein by reference. Depending on the therapeutic form of the system (e.g., intravenous injection versus oral tablet), the carrier may be in various forms.

Compounds described by formula (1) or formula (2) may be administered in the form of generally accepted oral compositions, such as tablets, coated tablets, gelatin capsules with hard and soft coating, emulsions or suspensions. Examples of vehicles which can be used for preparation of such compositions, include lactose, cornstarch or its derivatives, talc, stearic acid or its salts, etc. Acceptable vehicles for soft-coated gelatin capsules are, for example, vegetable oils, waxes, fats, semi-hard and liquid polyols, etc. In addition, pharmaceutical compounds may contain preservatives, solubilizers, stabilizers, wetting agents, emulsifiers, sweeteners, coloring agents, flavors, salts for changing osmotic pressure, buffers, coating agents or antioxidants. They may also contain other substances, which possess valuable therapeutic properties. Preparative forms may represent typical standard dose and can be prepared using methods known in pharmacy.

Pharmaceutical formulations may be administered in the form of conventional oral compositions, such as tablets, coated tablets, gelatin capsules with hard and soft coating, emulsions or suspensions. Preferably, however, they have liquid forms, suitable for intravenous injections or for droppers. Examples of carriers which can be utilized for the manufacture of such compositions are lactose, maize starch or its derivatives, talc, stearic acid or its salts, etc. Acceptable carriers for gelatin capsules with a soft coating are, for example, vegetable oils, waxes, fats, semi-solid and liquid polyols, etc. In addition, pharmaceutical preparations may contain preservatives, solubilizers, stabilizers, wetting agents, emulsifiers, sweeteners, colorants, correctives, salts for altering osmotic pressure, buffers, coating agents or antioxidants. They may also contain other substances which have desirable therapeutic properties. Preparative forms may comprise the normal standard dose and may be prepared by methods well known in pharmacy. Suitable formulations can be found, e.g., in Remington's Pharmaceutical Sciences, supra, which is incorporated herein by reference.

### Exemplary Dosing Regimens

In order to prepare a pharmacological drug, one or more compounds described by formula (1) or formula (2) are mixed as an active ingredient with known in medicine pharmaceutically acceptable vehicle using methods adopted in pharmaceutics. Depending on the drug formulation, the vehicle may assume various forms.

According to the invention, the method for treatment and prevention of stresses, anxiety, neuroses, obsessive fears and their consequences is realized by administering to a patient a pharmacological drug containing an effective amount of a hydrogenated pyrido[4,3-b]indole such as dimebon described by formula (1) or formula (2) in the dose of 0.1-10 mg/kg of body weight at least once a day for the duration necessary to achieve therapeutic effect. This dose range of the pharmacological drug was confirmed by the authors using examples provided below based on recommendations for converting doses for animals and humans found in the book “Guidelines for experimental (pre-clinical) study of new pharmacological substances,” (in Russian), Moscow, Minzdrav RF (Ministry of Health of the Russian Federation), 2005, p. 207).

As used herein, unless clearly indicated otherwise, a compound or combination therapy of the invention may be administered to the individual by any available dosage form. In one variation, the compound or combination therapy is administered to the individual as a conventional immediate release dosage form. In one variation, the compound or combination therapy is administered to the individual as a sustained release form or part of a sustained release system, such as a system capable of sustaining the rate of delivery of a compound to an individual for a desired duration, which may be an extended duration, such as a duration that is longer than the time required for a corresponding immediate-release dosage form to release the same amount (e.g., by weight or by moles) of compound or combination therapy, and can be hours or days. A desired duration may be at least the drug elimination half life of the administered compound or combination therapy and may be about any of, e.g., at least about 6 hours or at least about 12 hours or at least about 24 hours or at least about 30 hours or at least about 48 hours or at least about 72 hours or at least about 96 hours or at least about 120 hours or at least about 144 or more hours, and can be at least about one week, at least about 2 weeks, at least about 3 weeks, at least about 4 weeks, at least about 8 weeks, or at least about 16 weeks or more.

The compound or combination therapy may be formulated for any available delivery route, whether immediate or sustained release, including an oral, mucosal (e.g., nasal, sublingual, vaginal, buccal or rectal), parenteral (e.g., intramuscular, subcutaneous, or intravenous), topical or transdermal delivery form. A compound or combination therapy may be formulated with suitable carriers to provide delivery forms, which may be but are not required to be sustained release forms, that include, but are not limited to: tablets, caplets, capsules (such as hard gelatin capsules and soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

The amount of compound, for example, a hydrogenated pyrido[4,3-b]indole such as dimebon or any of compounds 1 to 9, in a delivery form may be any effective amount, which may be from about 10 ng to about 1,500 mg or more of the single active ingredient compound of a monotherapy or of more than one active ingredient compound of a combination therapy. In one variation, a delivery form, such as a sustained release system, comprises less than about 30 mg of compound. In one variation, a delivery form, such as a single sustained release system capable of multi-day administration, comprises an amount of compound such that the daily dose of compound is less than about 30 mg of compound.

A treatment regimen involving a dosage form of compound, whether immediate release or a sustained release system, may involve administering the compound to the individual in dose of between about 0.1 and about 10 mg/kg of body weight, at least once a day and during the period of time required to achieve the therapeutic effect. In other variations, the daily dose (or other dosage frequency) of a hydrogenated pyrido[4,3-b]indole as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of dimebon is administered, such as a daily dosage that is less than about 0.1 mg/kg, which may include but is not limited to, a daily dosage of about 0.05 mg/kg.

The compound, including a hydrogenated pyrido[4,3-b]indole such as dimebon or any of compounds 1 to 9, may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosing frequency can be about a once weekly dosing. The dosing frequency can be about a once daily dosing. The dosing frequency can be more than about once weekly dosing. The dosing frequency can be less than three times a day dosing. The dosing frequency can be about three times a week dosing. The dosing frequency can be about a four times a week dosing. The dosing frequency can be about a two times a week dosing. The dosing frequency can be more than about once weekly dosing but less than about daily dosing. The dosing frequency can be about a once monthly dosing. The dosing frequency can be about a twice weekly dosing. The dosing frequency can be more than about once monthly dosing but less than about once weekly dosing. The dosing frequency can be intermittent (e.g., once daily dosing for 7 days followed by no doses for 7 days, repeated for any 14 day time period, such as about 2 months, about 4 months, about 6 months or more): The dosing frequency can be continuous (e.g., once weekly dosing for continuous weeks). Any of the dosing frequencies can employ any of the compounds described herein together with any of the dosages described herein, for example, the dosing frequency can be a once daily dosage of less than 0.1 mg/kg or less than about 0.05 mg/kg of dimebon.

In one variation, dimebon is administered in a dose of 5 mg once a day. In one variation, dimebon is administered in a dose of 5 mg twice a day. In one variation, dimebon is administered in a dose of 5 mg three times a day. In one variation, dimebon is administered in a dose of 10 mg once a day. In one variation, dimebon is administered in a dose of 10 mg twice a day. In one variation, dimebon is administered in a dose of 10 mg three times a day. In one variation, dimebon is administered in a dose of 20 mg once a day. In one variation, dimebon is administered in a dose of 20 mg twice a day. In one variation, dimebon is administered in a dose of 20 mg three times a day. In one variation, dimebon is administered in a dose of 40 mg once a day. In one variation, dimebon is administered in a dose of 40 mg twice a day. In one variation, dimebon is administered in a dose of 40 mg three times a day.

### Exemplary Kits

The invention further provides kits comprising one or more compounds as described herein. The kits may employ any of the compounds disclosed herein and instructions for use. The kits may include instructions directed to any of the methods or uses described or disclosed herein. In one variation, the instructions are directed to use of a hydrogenated pyrido[4,3-b]indole of formula (1) or a pharmaceutically acceptable salt thereof to treat anxiety or mood disorders. In another variation, the instructions are directed to use of a hydrogenated pyrido[4,3-b]indole of formula (2) or a pharmaceutically acceptable salt thereof to treat anxiety or mood disorders. In one variation, R¹ is selected from the group consisting of CH₃—, CH₃CH₂— and PhCH₂—; R² is selected from the group consisting of H—, PhCH₂—, and 6-CH₃-3-Py-(CH₂)₂-; and R³ is selected from the group consisting of H, CH₃— and Br—. In another variation, the compound is 2,8-dimethyl-5-[2-(6-methyl-3-pyridyl)-ethyl]-2,3,4,5-tetrahydro-1H-pyrido[4,3-b]indole (Dimebon). In one variation, the kit employs a hydrogenated pyrido[4,3-b]indole such as dimebon. In other variations, the kit comprises one or more of compounds 1 to 9. The compound may be formulated in any acceptable form. The kits may be used for any one or more of the uses described herein, and, accordingly, may contain instructions for any one or more of the stated uses (e.g., decreasing one more symptoms resulting from an anxiety or mood disorder, limiting the extent of disability resulting from such a disorder, and/or increasing the quality of life for individuals suffering from a mood or anxiety disorder).

Kits generally comprise suitable packaging. The kits may comprise one or more containers comprising any compound described herein, in unit dosage form or in multiple dosage form. Each component (if there is more than one component) can be packaged in separate containers or some components can be combined in one container where cross-reactivity and shelf life permit. The kit components can be supplied as liquids or powders. If supplied as powders, the kits may further comprise a pharmaceutically acceptable buffer or other solution for preparing a liquid formulation of the compound.

The kits may optionally include instructions, generally written instructions, although electronic storage media (e.g., magnetic diskette or optical disk) containing instructions are also acceptable, relating to the use of component(s) of the kit in methods of the present invention (e.g., methods of treating anxiety or mood disorders). The instructions included with the kit generally include, for example, information describing the components of the kit and methods of administering those components to an individual in need thereof.

The technical result which can be secured when implementing the invention is a significant improvement in patient quality of life, such as recovery or considerable reduction in stress levels, anxiety, neuroses, obsessive fears and their consequences, or other reduction of the serious consequences of anxiety or mood disorders. The possibility of implementing the invention with achievement of the stated object and securement of the technical result is confirmed, but not exhausted, by the following examples.

A possibility of implementation of the invention with realization of claimed application and achievement of the technical result is confirmed, but not limited to the following examples.

To evaluate the anxiolytic effect of Dimebon, basic certified methods were used as recommended by the Pharmacological Committee of the Ministry of Health of the Russian Federation for investigating substances demonstrating anxiolytic effect (Voronina, T. A. and Seredenin, S. B., “Methodical recommendations on studying tranquilizing (anxiolytic) effect of pharmacological substances,” in GUIDELINES FOR EXPERIMENTAL (PRE-CLINICAL) STUDY OF NEW PHARMACOLOGICAL SUBSTANCES (in Russian), Moscow, Minzdrav R F (Ministry of Health of the Russian Federation), 2005, p. 253-262).

### EXAMPLES

### Example 1

### Anxiolytic Activity of Dimebon in the Conflict Situation Test

Anxiolytic activity was evaluated using a basic conflict situation method described by Vogel, a known and commonly used test (Vogel, J. R., Beer, B., and Clody, D. E., “A simple and reliable conflict procedure for testing anti-anxiety agents,” PSYCHOPHARMACOLOGIA (Berlin), 1971, v. 21, p. 1-'7; Molodavkin, G. M., and Voronina, T. A., “Multi-channel setup for searching tranquilizers and studying mechanisms of their action using conflict situation method,” (in Russian), Eksperim. i klin. farmakol. (Exp. Clin. Pharmacol.), 1995, v. 58, No. 2, pp.54-56; and File, S. E., “Animal models of different anxiety states,” in GABAA RECEPTORS AND ANXIETY: FROM NEUROBIOLOGY TO TREATMENT, N.Y., Raven Press, 1995, p. 93-113). Such conflict situations most frequently lead to stress and neurotic diseases.

The tests were conducted using outbred white male rats with body weights between 220 g and 250 g. Before beginning the test, experimental animals were randomly separated into groups of at least 10 rats. The conflict situation was created by suppressing the drinking reflex during water consumption from the drinking tube using algesic electric stimulus (i.e., electric shock causing pain). Thus, the Vogel conflict method is based on the conflict of two motivations: drinking and defensive. Thus, the animal fears punishment when attempting to satisfy its thirst, and that fear suppresses typical animal behavior. The animal cannot satisfy its thirst, creating an imbalance between what is desirable and what is real, thereby resulting in a stressful situation, accompanied by anxiety and fear of receiving an additional algesic electric stimulus. If a test compound has anxiolytic effect, it will enable the animal to overcome anxiety and fear of the punishment factor, restoring the ability to drink despite the possibility of receiving additional algesic electric stimuli, measured as an increase in incidents of punishable responses, i.e., drinking, despite the continued risk of receiving algesic electric stimuli.

The experimental setup consists of three parts: 4 experimental chambers, an electronic unit and a counting device. The experimental chamber measures 275×275×450 mm and is made of Plexiglas. It is installed on a standard electrode floor made of 4 mm diameter stainless steel rods spaced by 8-10 mm apart. Attached to the side wall of each chamber is a standard drinking tube attached to a glass container including a stainless steel nipple. In the device, the drinking tube is installed within common space of the chamber, not in the darkened section. The nipple extends 2 cm into the chamber at a height of 5 cm from the floor. The reason for such arrangement is that once exposed to a new environment, the animals instinctively attempt to hide in the dark section, and therefore may accidentally find a drinking tube, not as a consequence of purposeful search to satisfy the motivation. The electrode floor and the nipple of the drinking tube are connected to the electronic unit. The electronic unit contains current stabilizers (one per channel, which provides the possibility of independent current regulation), output signal generators for the counting device and delay generators for supplying punishing current to the drinking tube during the day of the experiment. That arrangement permits registration of non-punishable drinkings while establishing a drinking habit (trainings without supplying current to the drinking tubes), administration of a punishment current, and recordation of signals from punishable drinkings during the experiment.

The counting device enables registration of non-punishable drinkings during training and punishable drinkings during the experiment. Registration of readings was conducted using Pentium III personal computer with a 550 MHz C.P.U., a special device for converting output signals from the electronic unit into standard pulses suitable for inputting to the computer via a serial port, and a program written in BASIC (Beginner's All-Purpose Symbolic Instruction Code) for recording events and time intervals into disc files. The data accumulated in these files was later analyzed with the statistical package Statistica® for Windows®.

The experiment was conducted for 3 days. On the first day, the animals were completely deprived of water. On the second day, i.e., after 24-hours without water, animals were allowed to establish a habit of taking water from the drinking tube. To achieve this, animals were placed in the experimental chamber for 5 minutes. Typically, each animal surveyed the chamber and after a period of time found a drinking tube and started to drink. On the second day, a weak current of 50 μA was applied to the drinking tube and the chamber floor. That current was weak enough that it could not be felt by the rats, meaning that those drinkings were non-punishable. Thus, their number indicated how pronounced the drinking motivation was. On the third day, the animals were again placed in the experimental chamber, this time for 10 minutes. This time, 10 seconds after the first drinking a direct current of 0.25 mA was applied to the nipple of the drinking tubes and the electrode floor of the chamber. At that level, each drink was punishable, so the animals experienced high stress.

Thus, on day 3, in order to satisfy their thirst, the animals would have to overcome anxiety and fear developed as a result of the punishment. A significant increase in the number of punishable drinkings from the drinking tube (i.e., drinkings despite receipt of an algesic stimulus) in the experimental group compared to the control groups during 10 minutes of registration was considered to be an indication that the drug had anxiolytic effect.

Dimebon (2,8-dimethyl-5-[2-(6-methyl-3-pyridyl)ethyl]-2,3,4,5-tetrahydro-1H-pyrido[4,3-b]indole) was administered intraperitoneally in the doses of 0.05 mg/kg, 0.1 mg/kg, 2 mg/kg, and 5 mg/kg forty minutes before beginning the experiment. The reference drug Diazepam® (Seduxen®, manufactured by Gedeon Richter-Rus, Budapest, Hungary) was administered intraperitoneally at a therapeutic dose of 2 mg/kg forty minutes before beginning the experiment. Rats in the control group received 0.2 ml or water per 100 g body mass injected intraperitoneally.

Behavior of the control group animals after receiving an unexpected algesic stimulus when attempting to satisfy the normal drinking reflex (i.e., thirst) was characterized by expressed stress. At first, animals demonstrate distinctive tension, freezing and stillness, but then, since the animal received only a single algesic stimulus, the uncertainty of receiving another shock was not yet high, the animal attempted to take another water and received another algesic stimulus. From that moment on, the rat begins to understand that it is confronted with a conflict situation. That is, the necessity to satisfy the sense of thirst confronts the fear of receiving an algesic stimulus while drinking. Nevertheless, despite the fact that they received an algesic stimulus, rats of the control group approached the drinking tube in an attempt to satisfy their thirst and received punishable drinkings (average reading—143.25) over 10 minutes of registration. That is considered a major behavioral indicator in the conflict situation (see Table 1).

Table 1 shows the number of incidents of punishable drinking, the main indicator of the behavior in conflict situation characterizing anxiolytic effect of Dimebon in Vogel's conflict situation test, which increases with increasing dosage of Dimebon.

TABLE 1

| | | No. of punishable |
|---|---|---|
| Substances | Dose (mg/kg) | drinkings |
| Control (distilled water) | 0 (H₂O) | 143.25 ± 15.39 |
| Dimebon | 0.05 | 171.31 ± 15.67 |
| Dimebon | 0.1 | 261.18 ± 29.74* |
| Dimebon | 2.0 | 354.25 ± 32.49* |
| Dimebon | 5.0 | 405.33 ± 26.35* |
| Diazepam ® | 2.0 | 372.71 ± 27.81* |

*significance of the difference as compared to control at P < 0.05. (P—probability of variations between the control and experimental groups is considered significant if P ≦ 0.05).

It was established that Dimebon has a clear anxiolytic effect on animal behavior in the conflict situation. Under the effect of the drug, a statistically significant increase in punishable drinkings was observed. Animals continued to attempt to get water despite receiving algesic electric stimuli when doing so. Significant effect was observed when using Dimebon in the wide dose ranges from 0.1 to 5 mg/kg (see Table 1). The anxiolytic effect of Dimebon is dose-dependent meaning that drug activity increased with the increase in dose.

Reference benzodiazepine anxiolytic (Diazepam®) also caused significant anxiolytic effect at a dose of 2 mg/kg forty minutes after administration, which was similar to Dimebon at the same dose (see Table 1). Based on the strength of anxiolytic effect in the conflict situation, Dimebon is comparable in anxiolytic activity to Diazepam®, although use of Diazepam® at a therapeutic dose of 2 mg/kg leads to sedation, reduction in motion activity and impaired coordination.

Hence, these data confirm the fact that Dimebon demonstrates pronounced dose-dependent anxiolytic effect at a wide range of doses (0.1-5.0 mg/kg) using the basic Vogel conflict situation test. Furthermore, the anxiolytic effect of dimebon comparesfavorably to the reference drug Diazepam®, although Dimebon lacks the sedative and muscle relaxant side effects of Diazepam®.

### Example 2

### Anxiolytic Effect of Dimebon Determined by the Elevated Plus-Maze Method

To evaluate anxiolytic effect of Dimebon, the widely used elevated plus-maze (“EPM”) model was also used. (Pellow, S., et al., “Validation of open:closed arm entries in elevated plus-maze as a measure of anxiety in the rat,” Neurosci. Meth. J., 1985, No. 14, pp. 149-167; Voronina, T. A., et al., “Guideline for experimental (pre-clinical) study of new pharmacological sustances,” (in Russian), Moscow, Meditsyna, 2005, pp. 253-263). The model is based on stress and fear, which appear in animals while displaying orientation-investigative behavior and burrowing reflex under the complicated conditions of elevated plus-maze (novelty of surroundings, fear of height and illumination).

EPM for rats is performed in a chamber consisting of four compartments formed by the crossing of two strips measuring 50×10 cm. Two opposing compartments have vertical walls 40 cm high (protected dark arms), while two others (unprotected open bright arms) are free from protective walls. The maze is elevated 50 cm off the floor. At the center of the chamber where the two strips cross, there is a central platform measuring 10×10 cm. Rats were placed on the central platform with their tails facing an open bright arm. Time remaining on the central platform, time spent by the animals inside the open arms, and the number of entries into open and closed arms were recorded. The total observation time for each animal was 5 minutes. The main criterion of anxiolytic effect is the measure of time spent in the open bright arm of the setup. The number of crossings the central platform (number of entries into the dark and bright arms of the maze and the sum thereof) was used to estimate the effect of compounds on orientation-investigative behavior and motion activity of rats.

Experiments were conducted using white outbred male rats weighing 240 g to 280 g. Before beginning the test, experimental animals were randomly separated into groups of at least 10 rats. Animals of the experimental groups received Dimebon intraperitoneally at 0.1 mg/kg or 2.0 mg/kg, as well as a reference substance—Diazepam® at 2 mg/kg. Rats in the control group received 0.2 ml water per 100 g mass administered intraperitoneally. Data was recorded beginning forty minutes after administration of the compounds.

At a dose of 0.1 mg/kg, Dimebon considerably increased the main indicator of rat behavior during EPM time spent inside the open, dangerous arms of the maze (nearly five times as long as the control). In this case, a statistically significant increase was also observed with regard to the number of entries inside the bright arms without considerable change in the latent time spent on the central platform and the number of entries into the dark arms of the maze (see Table 2).

Table 2 shows the anxiolytic effect of Dimebon on rats in the elevated plus-maze method.

TABLE 2

| | | | Number of | Number of | Time in | |
|---|---|---|---|---|---|---|
| Animal | Doses, | Latent time | entries into | entries into | bright arms | Total number |
| group | (mg/kg) | (sec) | dark arms | bright arms | (sec) | of crossings |
| Control | 0 (H₂O) | 4.90 ± 0.67 | 5.80 ± 1.69 | 1.00 ± 0.45 | 5.20 ± 2.64 | 6.80 ± 2.01 |
| (distilled | | | | | | |
| water) | | | | | | |
| Dimebon | 0.1 | 3.43 ± 0.53 | 5.86 ± 1.53 | 1.86 ± 1.00* | 20.00 ± 7.75* | 7.71 ± 2.25 |
| | 2.0 | 2.93 ± 0.41 | 7.10 ± 3.21 | 4.25 ± 1.18* | 26.34 ± 8.12* | 11.60 ± 3.30* |
| Diazepam ® | 2.0 | 7.68 ± 2.0* | 2.20 ± 0.47* | 2.12 ± 1.08* | 25.6 ± 6.87* | 4.32 ± 1.01* |

*significance of the difference as compared to control at P < 0.05. (P—probability of variations between the control and experimental groups is considered significant if P ≦ 0.05).

That Dimebon changed animal behavior so significantly in the EPM demonstrates its stress-protective, anti-anxiety (i.e., anxiolytic) effect. Increasing the dose of

Dimebon to 2 mg/kg led to even more pronounced increase in those effects. This is supported by a considerable increase in time spent by the animals in the open arms of the maze under the action of the drug, as well as an increase of more than twice in the number of entries into those arms. The 2 mg/kg dose of Dimebon did not change the latent time spent on the central platform and only barely increased the number of entries into the dark protected arms of the maze, so that an increase in total number of crossings (by 2 times at P≦0.05) resulted mainly from an increase in the number of entries into the bright arms of the maze (see Table 2). Such a behavioral strategy in rats indicates that along with a clear anti-stress effect, Dimebon also causes improvement in orientation-investigative behavior of rats.

Like Dimebon, the reference drug Diazepam® at a dose of 2 mg/kg also demonstrated anxiolytic effect leading to a significant increase in the main indicator of the behavior—time spent by the rats in open arms of the maze. At the same time, with Diazepam® the animals demonstrated a statistically significant increase in latent time spent on the central platform and a decrease in the total number of crossings and the number of entries into the dark arms, indicating a disruption in orientation-investigative behavior as well as a sedative, depriming effect of the drug (see Table 2).

Thus, when used in the dose range of 0.1-2.0 mg/kg and in this test, Dimebon demonstrates an anxiolytic effect and optimizes the strategy of animal behavior under conditions of the elevated plus-maze method. In contrast, the anxiolytic effect of the reference substance Diazepam® is accompanied by a pronounced sedative effect.

### Example 3

### Anxiolytic Effect of Dimebon Under Stress in the Open Field Test

The “open field” test uses a method of creating stress based on a rat's fear of new surroundings, open space and bright illumination. This method was used to evaluate the anxiolytic effect of the claimed compounds (Voronina, T. A., and Seredenin, S. B., “Methodical recommendations concerning studying tranquilizing (anxiolytic) effect of pharmacological substances,” (in Russian), in GUIDELINE FOR EXPERIMENTAL (PRE-CLINICAL) STUDY OF NEW PHARMACOLOGICAL SUBSTANCES, Moscow, Minzdrav RF (Ministry of Health of the Russian Federation), 2005, p. 253-262; and File, S. E., “Animal models of different anxiety states,” in GABA RECEPTORS AND ANXIETY: FROM NEUROBIOLOGY TO TREATMENT, N.Y., Raven Press, 1995, p. 93-113.

An open field setup used in this study consisted of a one meter square box (1 m×1 m×1 m) with a clear top. The floor of the chamber was uniformly divided by lines into 9 squares, with 16 2.5 cm diameter holes. Before the experiment, rats were kept in the dark for 10 minutes, after which they were placed onto one of the peripheral squares of the open field. The animal was observed for 3 minutes. During the experiment the following information was recorded: the number of crossed squares on the periphery and in the center (separately), the number of vertical stands, the number of times holes were surveyed, and the number of exits to the center of the open field.

The main indicator of anxiolytic effect of the drugs was the measure of number of exits by the rat to the center of illuminated field. Increase or decrease in the number of horizontal or vertical movements reflected sedative or stimulating effect of the drug, while the number of holes surveyed reflects orientation-investigative behavior of the rat.

Experiments were conducted using white outbred male rats weighing 240 g to 280 g. Before the beginning of the test, experimental animals were randomly separated into groups containing at least 10 rats. Experimental animals were intraperitoneally injected with Dimebon at 0.1 mg/kg, 2.0 mg/kg, or 5.0 mg/kg, or with 2.0 mg/kg of Diazepam®. Rats in the control group received 0.2 ml water per 100 g mass administered intraperitoneally. Data was recorded beginning 40 minutes after administering the compounds.

In the control group, nine out of ten rats failed to come out to the center of the open field, which is an indication of a pronounced stress situation. In contrast, it was found that Dimebon at doses of 2.0 mg/kg and 5 mg/kg increased the number of exits to the center of the open illuminated field in concentration-dependent fashion (see Table 3).

Table 3 presents data showing the effect of Dimebon on rat behavior in the stress situation of the open field.

TABLE 3

| | | | | | Exits to |
|---|---|---|---|---|---|
| | | | | | the center |
| | | Horizontal | Vertical | Surveyed | of the |
| Substances | Doses | activity | activity | holes | field |
| Control | 0 (H₂O) | 18.7 ± 2.3 | 8.3 ± 2.7 | 14.2 ± 2.7 | 0.1 ± 0 |
| (distilled | | | | | |
| water) | | | | | |
| Dimebon | 0.1 mg/kg | 21.7 ± 5.9 | 7.8 ± 1.3 | 12.3 ± 1.7 | 0.8 ± 0.3 |
| Dimebon | 2.0 mg/kg | 20.3 ± 6.1 | 6.9 ± 3.2 | 11.2 ± 2.6 | 1.5 ± 0.2 |
| Dimebon | 5 mg/kg | 19.2 ± 2.4 | 9.3 ± 2.4 | 13.5 ± 1.9 | 2.2 ± 0.3 |
| Diazepam ® | 2 mg/kg | 10.2 ± 1.4 | 3.4 ± 1.3 | 6.5 ± 1.9 | 1.8 ± 0.3 |

*significance of the difference as compared to control at P < 0.05. (P—probability of variations between the control and experimental groups is considered significant if P ≦ 0.05).

The effect of Dimebon on increase in the number of exits to the center of the open illuminated field evidences the pronounced anxiolytic effect of the drug. Along with this, when used in the doses of 0.1, 2 and 5 mg/kg Dimebon did not cause a decrease in the number of horizontal and vertical movements of rats within open field as well as the number of surveyed holes (Table 3), which is indicative of the fact that the drug does not demonstrate sedative and muscle relaxation effects.

Like Dimebon, a dose of 2 mg/kg Diazepam® also caused anxiolytic effect in the open field test, which resulted in increase in the number of exits to the center of illuminated field. However, unlike Dimebon, Diazepam® at a dose of 2 mg/kg significantly suppressed horizontal and vertical motion activity within the open field, and also reduced the number of surveyed holes, showing the sedative effect of the drug (see Table 3).

Thus, at doses of 2 and 5 mg/kg, Dimebon demonstrated significant, pronounced anxiolytic effect in the open field test, and its activity is similar to that of Diazepam®. The essential advantage of Dimebon over Diazepam® is that the former, when used in therapeutic doses, does not demonstrate a sedative, muscle relaxation effect. Hence, the anxiolytic effect of Dimebon is observed without interference by the sedative and muscle relaxation effects, unlike Diazepam®, for which the anxiolytic effect is always accompanied by behavioral suppression.

### Example 4

### Effect of Dimebon on Behavior in Mice with Genetically Determined Elevated Level of Anxiety using the Senescence-Accelerated Mouse P/10 (SAM-P/10) Under Conditions of Elevated Plus-Maze

One of the modern methods of studying the anxiolytic effect of drugs uses a mouse strain with an increased level of anxiety. This study utilized mice of the SAM-P/10 line (Takeda, T., et al., “Senescence-Accelerated Mouse (SAM): A Novel Murine Model of Accelerated Senescence,” J. Amer. Geriatr. Soc., 1991, v. 39, pp. 911-919) weighing between 26 g and 31 g, in which, among other changes, a genetically determined anxiety is observed. Animals of this line demonstrate anxiety, stress, circadian rhythm impairment, accumulation of cerebral β-amyloid, balance impairment, neuromediator systems, etc. Those symptoms actively accumulate beginning from the age of 6-months (accelerated aging) (Miyamoto, M., “Indicators of age-related behavioral changes in Senescence-Accelerated Mouse SAMP8 and SAMP10,” Exp. Gerontol., 1997, v. 32, pp. 139-148; and Shimada, A., et al., “Age-related deterioration in conditional avoidance task in the SAM-P/10 mouse, an animal model of spontaneous brain atrophy,” Brain Res., 1993, v. 608, pp. 266-272).

This study utilized SAM-P/10 (Senescence-Accelerated Mouse P10) mice in two age groups—3-months and 11-months old. A group of 3-month old animals and a group of 11-month old animals were each divided into three subgroups: one group received Dimebon at a dose of 0.05 mg/kg, the second group received Dimebon at a dose of 2 mg/kg, and the third group received physiological saline. All substances were administered intraperitoneally 40 minutes prior to testing in the amount of 0.1 ml per 10 g of mouse weight. This study was conducted during the first half of the day from 10:00 a.m. to 2:00 p.m. Evaluation of the anxiety level in mice was performed using the elevated plus-maze (EPM) method (Pellow S., et al., “Validation of open:closed arm entries in elevated plus-maze as a measure of anxiety in the rat,” Neurosci. Meth. J., 1985, No. 14, pp. 149-167; Voronina, T. A., et al., “Guideline for experimental (pre-clinical) study of new pharmacological substances,” Moscow, Meditsyna, 2005, pp. 253-263).

EPM for mice is performed in a chamber consisting of four compartments formed by the crossing of two strips measuring 45×5 cm. Two opposing compartments have vertical walls 30 cm high (protected dark arms), while two others (unprotected open bright arms) are free from protective walls. The maze is elevated 30 cm off the floor. Where the two strips cross, there is a central platform measuring 5×5 cm. Mice were placed on the central platform with their tails facing the bright arm. Recorded behaviors included latent time remaining on the central platform, the time spent by the animals inside open arms, and the number of entries into open and closed arms. The total observation time for each animal was 5 minutes. Time spent by mice in the open arms of the maze was used as a main indicator of anxiety level.

It was found that in comparison with the control 3-month old animals of the SAM-P/10 line, control 11-month old SAM-P/10 mice demonstrated a considerable decrease in the main indicator (by more than 6 times)—time spent in the open bright arms of the maze, as well as an increase in latent time and a considerable, statistically significant decrease in the number of entries into the dark and bright arms of the maze (see Table 4). Such behavior in the 11-month old mice of the SAM-P/10 line under conditions of elevated plus-maze reflects the development of anxiety and stress syndrome in mice. When used at a dose of 0.05 mg/kg in 11-month old mice of the SAM-P/10 line, Dimebon doubled the main indicator of mice behavior inside EPM—time spent in the open, unprotected arms of the maze. In addition, the drug also improved other indicators of stressed behavior: it increased the number of entries into the bright arms and the total number of crossings (see Table 4). The results obtained confirm that Dimebon has anxiolytic effect in rats and mice.

Table 4 shows the effect of Dimebon on the behavior of SAM-P/10 mice, which have an increased level of anxiety as measured by the elevated plus-maze (EPM) method as applied to two groups of mice at different ages—3 months and 11 months.

TABLE 4

| | | | Number of | Number of | Time in | Total |
|---|---|---|---|---|---|---|
| Animal | Doses | Latent time | entries into | entries into | bright arms | number of |
| group | (mg/kg) | (sec) | dark arms | bright arms | (sec) | crossings |
| Control (3 | 0 (H₂O) | 2.2 ± 0.3 | 15.0 ± 2.4 | 7.7 ± 1.2 | 26.0 ± 4.6 | 22.7 ± 2.7 |
| months) | | | | | | |
| Control | 0 (H₂O) | 9.80 ± 1.6* | 4.9 ± 1.2* | 1.0 ± 0.4* | 4.2 ± 1.1* | 8.0 ± 2.1* |
| (11 | | | | | | |
| months) | | | | | | |
| Dimebon | 0.05 | 7.5 ± 1.5 | 5.86 ± 1.53 | 1.9 ± 1.0* | 8.7 ± 3.3* | 11.7 ± 1.5* |
| (11 | 2.0 | 6.0 ± 1.4 | 7.10 ± 3.21 | 4.2 ± 1.1* | 17.7 ± 5.2* | 18.1 ± 5.3* |
| months) | | | | | | |

*significance of the difference as compared to control at P < 0.05. (P—probability of variations between the control and experimental groups is considered significant if P ≦ 0.05).
# - indicates significant difference in anxiety signs between young and old mice, who have developed signs of stress and anxiety.

Increasing the dose of Dimebon to 2 mg/kg significantly increased its anxiolytic effect. Thus, under the action of Dimebon, 11-month old mice of the SAM-P/10 line demonstrated more than 4-fold increase in the time spent in the dangerous, open arms of the maze as well as in the number of entries into them; there was also an increase in the sum of crossings while latent time decreased. A combination of these changes in the behavior of mice when using Dimebon evidences a pronounced anti-stress and anti-anxiety effect of the drug.

Thus, Dimebon demonstrated a clear anxiolytic effect in the experiments with mice of the SAM-P/10 line with a genetically determined high level of anxiety and stress, which can be clearly seen using the elevated plus-maze model. The effect of Dimebon was dose-dependent meaning that a positive anxiolytic effect of the drug increased with the increase in dose from 0.05 mg/kg to 2 mg/kg.

The experiments conducted leads to the conclusion that Dimebon demonstrates anxiolytic effect when administered intraperitoneally in the experiments with animals in the dose range of 0.05mg/kg to 5 mg/kg. The effect of the drug was revealed using the basic experimental models of anxiety and stress—conflict situation, elevated plus-maze and open field. Dimebon proved effective as an anxiolytic in mice of the SAM-P/10 line with genetically determined increase in the level of stress, anxiety and retardation. A considerable advantage of Dimebon over Diazepam® is the absence of depriming, sedative and muscle relaxation effects when used in doses with pronounced anxiolytic effect.

Dimebon is an anxiolytic of a new type, which demonstrates anti-stress, anti-anxiety and tranquilizing effects with no side effects typical for traditional anxiolytics (sedation, muscle relaxation, memory impairment, drug dependence)

Dimebon may be used in psychiatric practice for treatment and prevention of stresses, anxiety, neuroses, obsessive fears and their consequences, accompanied by anxiety, fear, emotional stress, tension, asthenia. Dimebon can be used not only in psychiatry, but also in different fields of medicine in case of various diseases accompanied by emotional stress, anxiety and fears, as well as panic conditions.

Dimebon can be used to treat stress and anxiety developing in healthy people under psychological and traumatic factors and in different extreme situations, and specifically, in humans, whose activity is associated with working under extreme and complicated conditions (special services workers, military personnel, rescuers, sportsmen, mountain-climbers, etc.).

All references, publications, patents, and patent applications disclosed herein are hereby incorporated herein by reference in their entireties.

1. A method for treating anxiety or mood disorders in an individual in need thereof comprising administering a therapeutically effective amount of hydrogenated pyrido[4,3-b]indole of the formula (1) or a pharmaceutically acceptable salt thereof 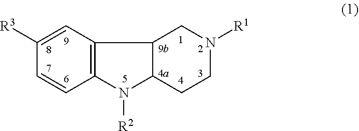
wherein R¹ is selected from the group consisting of CH₃—, CH₃CH₂— and PhCH₂—;
R² is selected from the group consisting of H—, PhCH₂—, and 6-CH₃-3-Py-(CH₂)₂-; and
R³ is selected from the group consisting of H, CH₃— and Br—.

2. The method of claim 1, wherein R¹ is CH₃—, R² is H—, and R³ is CH₃—.

3. The method of claim 1, wherein the pharmaceutically acceptable salt is a pharmaceutically acceptable acid salt.

4. The method of claim 1, wherein the anxiety or mood disorder is selected from the group consisting of generalized anxiety disorder, panic disorder, phobias, social anxiety disorder, obsessive-compulsive disorder, post-traumatic stress disorder, and separation anxiety.

5. A method for treating anxiety or mood disorders in an individual in need thereof comprising administering a therapeutically effective amount of hydrogenated pyrido[4,3-b]indole of formula (2) or a pharmaceutically acceptable salt thereof, 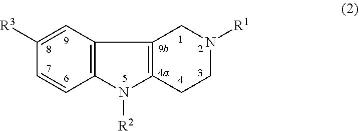
wherein R¹ is selected from the group consisting of CH₃—, CH₃CH₂— and PhCH₂—;
R² is selected from the group consisting of H—, PhCH₂—, and 6-CH₃-3-Py-(CH₂)₂-; and
R³ is selected from the group consisting of H, CH₃— and Br—.

6. The method of claim 5, wherein R¹ is CH₃CH₂— or PhCH₂—; R² is H—; and R³— is H—.

7. The method of claim 5, wherein R¹ is CH₃—; R² is PhCH₂—; and R³ is CH₃—.

8. The method of claim 5, wherein R¹ is CH₃—, R² is 6-CH₃-3-Py-(CH₂)₂-, and R³— corresponds to is H—.

9. The method of claim 5, wherein R¹ is CH₃—, R² is 6-CH₃-3-Py-(CH₂)₂-, and R³— CH₃—.

10. (canceled)

11. The method of claim 5, wherein R¹ is CH₃—, R² is H—, and R³ is Br—.

12. The method of claim 5, wherein the pharmaceutically acceptable salt is a pharmaceutically acceptable acid salt.

13. The method of claim 5, wherein the hydrogenated pyrido[4,3-b]indole is 2,8-dimethyl-5-[2-(6-methyl-3-pyridyflethyl]-2,3,4,5-tetrahydro-1H-pyrido[4,3-b]indole dihydrochloride.

14. The method of claim 5, wherein the anxiety or mood disorder is selected from the group consisting of generalized anxiety disorder, panic disorder, phobias, social anxiety disorder, obsessive-compulsive disorder, post-traumatic stress disorder, and separation anxiety.

15. A pharmaceutical composition having anxiolytic effect, comprising an active compound and a pharmaceutically acceptable carrier, wherein the active compound comprises an effective amount of a compound of formula (1) or formula (2) or a pharmaceutically acceptable salt thereof: 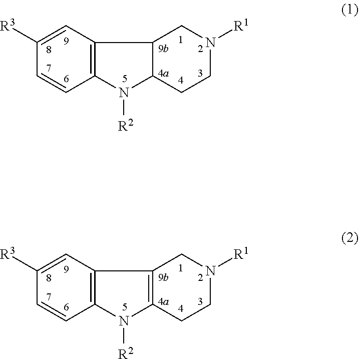
wherein R¹ is selected from the group consisting of CH₃—, CH₃CH₂— and PhCH₂—;
R² is selected from the group consisting of H—, PhCH₂—, and 6-CH₃-3-Py-(CH₂)₂-; and
R³ is selected from the group consisting of H, CH₃— and Br—.

16. The pharmaceutical composition of claim 15, wherein the active compound is an acid salt of 2,8-dimethyl-5-[2-(6-methyl-3-pyridyl)ethyl]-2,3,4,5-tetrahydro-1H-pyrido[4,3-b]indole.

17. A method of treating and preventing anxiety or mood disorders, comprising administering to the patient a composition containing an effective amount of a compound of formula (1) or formula (2) or a pharmaceutically acceptable salt thereof: 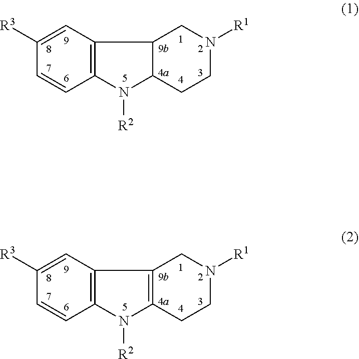
wherein R¹ is selected from the group consisting of CH₃—, CH₃CH₂— and PhCH₂—;
R² is selected from the group consisting of H—, PhCH₂—, and 6-CH₃-3-Py-(CH₂)₂-; and
R³ is selected from the group consisting of H, CH₃— and Br—; and
wherein the effective amount is administered at a dose between 0.1 mg/kg and 10 mg/kg of body weight at least once a day for a duration necessary to achieve therapeutic effect.

18. The method of claim 17, wherein the anxiety or mood disorder is selected from the group consisting of generalized anxiety disorder, panic disorder, phobias, social anxiety disorder, obsessive-compulsive disorder, post-traumatic stress disorder, and separation anxiety.

19. The method of claim 18, wherein the compound is 2,8-dimethyl-5-[2-(6-methyl-3-pyridyl)ethyl]-2,3,4,5-tetrahydro-1H-pyrido[4,3-b]indole.

20. A kit comprising:
(a) a hydrogenated pyrido[4,3-b]indole of formula (2) or a pharmaceutically acceptable salt thereof, 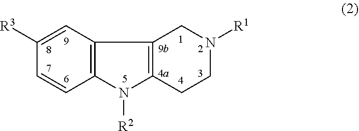
wherein:
R¹ is selected from the group consisting of CH₃—, CH₃CH₂— and PhCH₂—;
R² is selected from the group consisting of H—, PhCH₂—, and 6-CH₃-3-Py-(CH₂)₂-; and
R³ is selected from the group consisting of H, CH₃— and Br—; and
(b) instructions for use in treatment of anxiety or mood disorders.

21. The kit of claim 20, wherein the hydrogenated pyrido[4,3-b]indole is 2,8-dimethyl-5-[2-(6-methyl-3-pyridyl)ethyl]-2,3,4,5-tetrahydro-1H-pyrido[4,3-b]indole dihydrochloride.
